SEQUENCE LISTING (200 sequences)

SEQ ID NO: 1 - DNA, 24 bp, Artificial
  Alu#1 ACE 5′Primer
ctggagaccactcccatcctttct

SEQ ID NO: 2 - DNA, 25 bp, Artificial
  Alu#1 ACE 3′Primer
gatgtggccatcacattcgtcagat

SEQ ID NO: 3 - DNA, 25 bp, Artificial
  Alu#2 APO 5′Primer
aagtgctgtaggccatttagattag

SEQ ID NO: 4 - DNA, 25 bp, Artificial
  Alu#2 APO 3′Primer
agtcttcgatgacagcgtatacaga

SEQ ID NO: 5 - DNA, 20 bp, Artificial
  Alu#3 B65 5′Primer
atatcctaaaagggacacca

SEQ ID NO: 6 - DNA, 20 bp, Artificial
  Alu#3 B65 3′Primer
aaaatttatggcatgcgtat

SEQ ID NO: 7 - DNA, 30 bp, Artificial
  Alu#4 COL3A1 5′Primer
acctgcagcaccaggaggtcctggagggcc

SEQ ID NO: 8 - DNA, 25 bp, Artificial
  Alu#4 COL3A1 3′Primer
gagtcctttagaaggatatgctctg

SEQ ID NO: 9 - DNA, 20 bp, Artificial
  Alu#5 HS2.43 5′Primer
actccccaccaggtaatggt

SEQ ID NO: 10 - DNA, 20 bp, Artificial
  Alu#5 HS2.43 3′Primer
agggccttcatccagtttgt

SEQ ID NO: 11 - DNA, 20 bp, Artificial
  Alu#6 HS4.32 5′Primer
gtttattgggctaacctggg

SEQ ID NO: 12 - DNA, 25 bp, Artificial
  Alu#6 HS4.32 3′Primer
tgaccagctaacttctactttaacc

SEQ ID NO: 13 - DNA, 20 bp, Artificial
  Alu#7 HS4.65 5′Primer
tgaagccaatggaaagagag

SEQ ID NO: 14 - DNA, 21 bp, Artificial
  Alu#7 HS4.65 3′Primer
acaggagcatctaaaccttgg

SEQ ID NO: 15 - DNA, 26 bp, Artificial
  Alu#8 HS4.75 5′Primer
cagcattacatacaatagttaggagc

SEQ ID NO: 16 - DNA, 25 bp, Artificial
  Alu#8 HS4.75 3′Primer
gtgatatttgtctttctgtacctgg

SEQ ID NO: 17 - DNA, 23 bp, Artificial
  Alu#9 PV92 5′Primer
aactgggaaaatttgaagaaagt

SEQ ID NO: 18 - DNA, 25 bp, Artificial
  Alu#9 PV92 3′Primer
tgagttctcaactcctgtgtgttag

SEQ ID NO: 19 - DNA, 21 bp, Artificial
  Alu#10 Sb22777/Sb19.12 5′Primer
ttaacatccctgcaacccatc

SEQ ID NO: 20 - DNA, 22 bp, Artificial
  Alu#10 Sb22777/Sb19.12 3′Primer
gattatagtcaccctgttgtgc

SEQ ID NO: 21 - DNA, 25 bp, Artificial
  Alu#11 Sb23467/Sb19.3 5′Primer
tctaggcccagatttatggtaactg

SEQ ID NO: 22 - DNA, 24 bp, Artificial
  Alu#11 Sb23467/Sb19.3 3′Primer
aagcacaattggttattttctgac

SEQ ID NO: 23 - DNA, 25 bp, Artificial
  Alu#12 TPA25 5′Primer
gtaagagttccgtaacaggacagct

SEQ ID NO: 24 - DNA, 25 bp, Artificial
  Alu#12 TPA25 3′Primer
ccccaccctaggagaacttctcttt

SEQ ID NO: 25 - DNA, 22 bp, Artificial
  Alu#13 Ya5NBC102 5′Primer
tcccatttctctagacctgctg

SEQ ID NO: 26 - DNA, 24 bp, Artificial
  Alu#13 Ya5NBC102 3′Primer
cccataacaggtcttcatatttcc

SEQ ID NO: 27 - DNA, 24 bp, Artificial
  Alu#14 Ya5NBC120 5′Primer
ggaccacatgactgagtgtaaagt

SEQ ID NO: 28 - DNA, 24 bp, Artificial
  Alu#14 Ya5NBC120 3′Primer
gaggtggcctcttaaccataattc

SEQ ID NO: 29 - DNA, 27 bp, Artificial
  Alu#15 Ya5NBC123 5′Primer
atcaagttgacactcagtattcaccac

SEQ ID NO: 30 - DNA, 27 bp, Artificial
  Alu#15 Ya5NBC123 3′Primer
ctagtctgcagaactgtgagaaatgta

SEQ ID NO: 31 - DNA, 26 bp, Artificial
  Alu#16 Ya5NBC132 5′Primer
ctcgtgattcacagaagtgttgtaag

SEQ ID NO: 32 - DNA, 25 bp, Artificial
  Alu#16 Ya5NBC132 3′Primer
cggggttcatccttaatacatacat

SEQ ID NO: 33 - DNA, 23 bp, Artificial
  Alu#17 Ya5NBC135 5′Primer
attaagctcatggtaaccagcac

SEQ ID NO: 34 - DNA, 26 bp, Artificial
  Alu#17 Ya5NBC135 3′Primer
gactctcctctctggattagaaacag

SEQ ID NO: 35 - DNA, 25 bp, Artificial
  Alu#18 Ya5NBC147 5′Primer
tagctgggggaggtagataataaac

SEQ ID NO: 36 - DNA, 25 bp, Artificial
  Alu#18 Ya5NBC147 3′Primer
aaatatcaccttatcagtgggacct

SEQ ID NO: 37 - DNA, 25 bp, Artificial
  Alu#19 Ya5NBC148 5′Primer
acaagatgacagatgtaaacccaac

SEQ ID NO: 38 - DNA, 25 bp, Artificial
  Alu#19 Ya5NBC148 3′Primer
aaggtgttgtcagactaatctatcg

SEQ ID NO: 39 - DNA, 25 bp, Artificial
  Alu#20 Ya5NBC150 5′Primer
aaatggagacacagaggtgtaaaga

SEQ ID NO: 40 - DNA, 25 bp, Artificial
  Alu#20 Ya5NBC150 3′Primer
cccaaactgcatatttaaagggtag

SEQ ID NO: 41 - DNA, 25 bp, Artificial
  Alu#21 Ya5NBC157 5′Primer
catacgttaaatcactcggtactca

SEQ ID NO: 42 - DNA, 25 bp, Artificial
  Alu#21 Ya5NBC157 3′Primer
tcagaaaagtatacaggtgatgtgc

SEQ ID NO: 43 - DNA, 24 bp, Artificial
  Alu#22 Ya5NBC159 5′Primer
gagggtctttcctaggttttgttt

SEQ ID NO: 44 - DNA, 25 bp, Artificial
  Alu#22 Ya5NBC159 3′Primer
atgtcaggtgtcccttatggagtat

SEQ ID NO: 45 - DNA, 25 bp, Artificial
  Alu#23 Ya5NBC171 5′Primer
tctagaattacaagtgcaagccatc

SEQ ID NO: 46 - DNA, 25 bp, Artificial
  Alu#23 Ya5NBC171 3′Primer
cttctcatccctgctaacataacat

SEQ ID NO: 47 - DNA, 24 bp, Artificial
  Alu#24 Ya5NBC182 5′Primer
gaaggactatgtagttgcagaagc

SEQ ID NO: 48 - DNA, 22 bp, Artificial
  Alu#24 Ya5NBC182 3′Primer
aacccagtggaaacagaagatg

SEQ ID NO: 49 - DNA, 25 bp, Artificial
  Alu#25 Ya5NBC208 5′Primer
aataccttgtacatcttcaccccta

SEQ ID NO: 50 - DNA, 22 bp, Artificial
  Alu#25 Ya5NBC208 3′Primer
tctctctgctgcacagtttgtt

SEQ ID NO: 51 - DNA, 20 bp, Artificial
  Alu#26 Ya5NBC212 5′Primer
catttggcgcaagtggtatt

SEQ ID NO: 52 - DNA, 19 bp, Artificial
  Alu#26 Ya5NBC212 3′Primer
atcccaaagaaacccacga

SEQ ID NO: 53 - DNA, 21 bp, Artificial
  Alu#27 Ya5NBC216 5′Primer
gatgtgaccctggcttgtaaa

SEQ ID NO: 54 - DNA, 20 bp, Artificial
  Alu#27 Ya5NBC216 3′Primer
cagagtccctgtgcaaaatg

SEQ ID NO: 55 - DNA, 25 bp, Artificial
  Alu#28 Ya5NBC221 5′Primer
cagttttccatatacatgtgggttc

SEQ ID NO: 56 - DNA, 25 bp, Artificial
  Alu#28 Ya5NBC221 3′Primer
tagtgttaagaggcccattttctac

SEQ ID NO: 57 - DNA, 20 bp, Artificial
  Alu#29 Ya5NBC237 5′Primer
cccatggagggtctttccta

SEQ ID NO: 58 - DNA, 21 bp, Artificial
  Alu#29 Ya5NBC237 3′Primer
ctggaaaccatccttcacagt

SEQ ID NO: 59 - DNA, 25 bp, Artificial
  Alu#30 Ya5NBC239 5′Primer
cagctgagaactgtcacaaatagaa

SEQ ID NO: 60 - DNA, 24 bp, Artificial
  Alu#30 Ya5NBC239 3′Primer
atcaatgactgacttgtgctgagt

SEQ ID NO: 61 - DNA, 23 bp, Artificial
  Alu#31 Ya5NBC241 5′Primer
ggttccaatagagagcaacagaa

SEQ ID NO: 62 - DNA, 20 bp, Artificial
  Alu#31 Ya5NBC241 3′Primer
accttaagctttcccccaga

SEQ ID NO: 63 - DNA, 21 bp, Artificial
  Alu#32 Ya5NBC242 5′Primer
aacaaaattccctttcctcca

SEQ ID NO: 64 - DNA, 20 bp, Artificial
  Alu#32 Ya5NBC242 3′Primer
ggcaatctgaccttgggtaa

SEQ ID NO: 65 - DNA, 27 bp, Artificial
  Alu#33 Ya5NBC27 5′Primer
ctgaatacaggtatcactgaacagaac

SEQ ID NO: 66 - DNA, 29 bp, Artificial
  Alu#33 Ya5NBC27 3′Primer
acagtgtaaagtctaacctaccagaggat

SEQ ID NO: 67 - DNA, 20 bp, Artificial
  Alu#34 Ya5NBC311 5′Primer
tcttggcaaggagatgtgaa

SEQ ID NO: 68 - DNA, 20 bp, Artificial
  Alu#34 Ya5NBC311 3′Primer
aatcacatccgagggtgtct

SEQ ID NO: 69 - DNA, 20 bp, Artificial
  Alu#35 Ya5NBC327 5′Primer
aggcaggttcaatgttcaaa

SEQ ID NO: 70 - DNA, 22 bp, Artificial
  Alu#35 Ya5NBC327 3′Primer
ttgtcttattgtgctggctaga

SEQ ID NO: 71 - DNA, 20 bp, Artificial
  Alu#36 Ya5NBC333 5′Primer
ggcatgctatcattcccaaa

SEQ ID NO: 72 - DNA, 25 bp, Artificial
  Alu#36 Ya5NBC333 3′Primer
ccaaacttctgtttgagagaatacg

SEQ ID NO: 73 - DNA, 22 bp, Artificial
  Alu#37 Ya5NBC335 5′Primer
tgggtactttggccttagagaa

SEQ ID NO: 74 - DNA, 25 bp, Artificial
  Alu#37 Ya5NBC335 3′Primer
ttcacagcattagagagagttgatg

SEQ ID NO: 75 - DNA, 20 bp, Artificial
  Alu#38 Ya5NBC345 5′Primer
gccatgagagtggtcagcat

SEQ ID NO: 76 - DNA, 21 bp, Artificial
  Alu#38 Ya5NBC345 3′Primer
agtctccaccatctctgctgt

SEQ ID NO: 77 - DNA, 20 bp, Artificial
  Alu#39 Ya5NBC347 5′Primer
catgcccattgctttacgtt

SEQ ID NO: 78 - DNA, 20 bp, Artificial
  Alu#39 Ya5NBC347 3′Primer
tggggtagatggactcatcc

SEQ ID NO: 79 - DNA, 20 bp, Artificial
  Alu#40 Ya5NBC351 5′Primer
ttcctcccctttttcctgtt

SEQ ID NO: 80 - DNA, 22 bp, Artificial
  Alu#40 Ya5NBC351 3′Primer
tgtcagtatgtaaacccatgct

SEQ ID NO: 81 - DNA, 20 bp, Artificial
  Alu#41 Ya5NBC354 5′Primer
gtagcttggcctgtgctctt

SEQ ID NO: 82 - DNA, 21 bp, Artificial
  Alu#41 Ya5NBC354 3′Primer
cctctgggctgagaaactctt

SEQ ID NO: 83 - DNA, 27 bp, Artificial
  Alu#42 Ya5NBC45 5′Primer
tagggtaaggaatatgtgctgctttag

SEQ ID NO: 84 - DNA, 24 bp, Artificial
  Alu#42 Ya5NBC45 3′Primer
gtctctgaacgactatgtgagcag

SEQ ID NO: 85 - DNA, 30 bp, Artificial
  Alu#43 Ya5NBC51 5′Primer
atattccagaagtttccttacatctagtgc

SEQ ID NO: 86 - DNA, 25 bp, Artificial
  Alu#43 Ya5NBC51 3′Primer
aaagctttaagtctccaccatctct

SEQ ID NO: 87 - DNA, 30 bp, Artificial
  Alu#44 Ya5NBC54 5′Primer
gtttatgtcagtaggagttttctcgtgtag

SEQ ID NO: 88 - DNA, 25 bp, Artificial
  Alu#44 Ya5NBC54 3′Primer
tcattgtatcatctgctgtacctgt

SEQ ID NO: 89 - DNA, 22 bp, Artificial
  Alu#45 Ya5NBC61 5′Primer
tgaaataatccagttggggaag

SEQ ID NO: 90 - DNA, 30 bp, Artificial
  Alu#45 Ya5NBC61 3′Primer
gtatatctctaccgagactcagtttttagc

SEQ ID NO: 91 - DNA, 27 bp, Artificial
  Alu#46 Ya5NBC96 5′Primer
tagatgagatagagccatcaaacactc

SEQ ID NO: 92 - DNA, 30 bp, Artificial
  Alu#46 Ya5NBC96 3′Primer
gtaccctgtgagaaaatattaggagctatg

SEQ ID NO: 93 - DNA, 21 bp, Artificial
  Alu#47 Yb8NBC106 5′Primer
tcacagcacaattcacaactg

SEQ ID NO: 94 - DNA, 20 bp, Artificial
  Alu#47 Yb8NBC106 3′Primer
ctgggttgcatttcatggta

SEQ ID NO: 95 - DNA, 24 bp, Artificial
  Alu#48 Yb8NBC120 5′Primer
cagtggatctccattttacctctc

SEQ ID NO: 96 - DNA, 23 bp, Artificial
  Alu#48 Yb8NBC120 3′Primer
ggaaaggtttcaggaagaaagtg

SEQ ID NO: 97 - DNA, 20 bp, Artificial
  Alu#49 Yb8NBC125 5′Primer
agccagaaaccctgaacaag

SEQ ID NO: 98 - DNA, 21 bp, Artificial
  Alu#49 Yb8NBC125 3′Primer
aaaggccccagaagtatacca

SEQ ID NO: 99 - DNA, 20 bp, Artificial
  Alu#50 Yb8NBC13 5′Primer
tctgggtttctctggtggac

SEQ ID NO: 100 - DNA, 20 bp, Artificial
  Alu#50 Yb8NBC13 3′Primer
ctggcaaatgctacccaagt

SEQ ID NO: 101 - DNA, 21 bp, Artificial
  Alu#51 Yb8NBC146 5′Primer
ctcttctctccaggaaacgtc

SEQ ID NO: 102 - DNA, 21 bp, Artificial
  Alu#51 Yb8NBC146 3′Primer
ggagctctgccttacactcaa

SEQ ID NO: 103 - DNA, 20 bp, Artificial
  Alu#52 Yb8NBC148 5′Primer
ccaggcctccatctttgata

SEQ ID NO: 104 - DNA, 20 bp, Artificial
  Alu#52 Yb8NBC148 3′Primer
tcacttttgggcatgtcaag

SEQ ID NO: 105 - DNA, 20 bp, Artificial
  Alu#53 Yb8NBC157 5′Primer
tatggttctcagccatcacg

SEQ ID NO: 106 - DNA, 20 bp, Artificial
  Alu#53 Yb8NBC157 3′Primer
attcttccccaaagggagtc

SEQ ID NO: 107 - DNA, 25 bp, Artificial
  Alu#54 Yb8NBC181 5′Primer
catgtaccttagaattccactctca

SEQ ID NO: 108 - DNA, 25 bp, Artificial
  Alu#54 Yb8NBC181 3′Primer
ccccaaagtttatagtctgttgtct

SEQ ID NO: 109 - DNA, 25 bp, Artificial
  Alu#55 Yb8NBC192 5′Primer
ctgctctaccctaggctcttctatc

SEQ ID NO: 110 - DNA, 25 bp, Artificial
  Alu#55 Yb8NBC192 3′Primer
gctcctctgcttttatgtgttctac

SEQ ID NO: 111 - DNA, 25 bp, Artificial
  Alu#56 Yb8NBC201 5′Primer
ggagaaaatgtaaggtttctagcac

SEQ ID NO: 112 - DNA, 25 bp, Artificial
  Alu#56 Yb8NBC201 3′Primer
accaatgcaactatctacactgaca

SEQ ID NO: 113 - DNA, 25 bp, Artificial
  Alu#57 Yb8NBC207 5′Primer
gtaatatgaggtgatgggggttact

SEQ ID NO: 114 - DNA, 25 bp, Artificial
  Alu#57 Yb8NBC207 3′Primer
ggtgaaagaagaacccctaagttat

SEQ ID NO: 115 - DNA, 20 bp, Artificial
  Alu#58 Yb8NBC227 5′Primer
aagaaaagggaagcctggag

SEQ ID NO: 116 - DNA, 20 bp, Artificial
  Alu#58 Yb8NBC227 3′Primer
cagtcatcaccagccatgag

SEQ ID NO: 117 - DNA, 20 bp, Artificial
  Alu#59 Yb8NBC237 5′Primer
gccaaaatcaactgccaaac

SEQ ID NO: 118 - DNA, 24 bp, Artificial
  Alu#59 Yb8NBC237 3′Primer
tgctgaggatagagctatagcaga

SEQ ID NO: 119 - DNA, 22 bp, Artificial
  Alu#60 Yb8NBC243 5′Primer
gaaccccatccattctcttaca

SEQ ID NO: 120 - DNA, 20 bp, Artificial
  Alu#60 Yb8NBC243 3′Primer
gtggcaaaatattggcgact

SEQ ID NO: 121 - DNA, 20 bp, Artificial
  Alu#61 Yb8NBC405 5′Primer
gcccatcccctattatagcc

SEQ ID NO: 122 - DNA, 20 bp, Artificial
  Alu#61 Yb8NBC405 3′Primer
accaaacccccatgacacta

SEQ ID NO: 123 - DNA, 22 bp, Artificial
  Alu#62 Yb8NBC412 5′Primer
caaagatggttgttgaggttga

SEQ ID NO: 124 - DNA, 20 bp, Artificial
  Alu#62 Yb8NBC412 3′Primer
cccagcaacttccccttaat

SEQ ID NO: 125 - DNA, 20 bp, Artificial
  Alu#63 Yb8NBC419 5′Primer
catctcctggcaacactgag

SEQ ID NO: 126 - DNA, 22 bp, Artificial
  Alu#63 Yb8NBC419 3′Primer
acaaagcaagggtatttacagc

SEQ ID NO: 127 - DNA, 20 bp, Artificial
  Alu#64 Yb8NBC420 5′Primer
aaatgcccaagtttcattgc

SEQ ID NO: 128 - DNA, 20 bp, Artificial
  Alu#64 Yb8NBC420 3′Primer
aactgccacagcgattcttt

SEQ ID NO: 129 - DNA, 20 bp, Artificial
  Alu#65 Yb8NBC435 5′Primer
tgaatgattgggactgggta

SEQ ID NO: 130 - DNA, 20 bp, Artificial
  Alu#65 Yb8NBC435 3′Primer
tggctggatgaactttcaca

SEQ ID NO: 131 - DNA, 20 bp, Artificial
  Alu#66 Yb8NBC437 5′Primer
ggcggtgatggtaaaacaac

SEQ ID NO: 132 - DNA, 20 bp, Artificial
  Alu#66 Yb8NBC437 3′Primer
cttccccaaggagcctttta

SEQ ID NO: 133 - DNA, 20 bp, Artificial
  Alu#67 Yb8NBC441 5′Primer
ctcctggcatgtcttcaggt

SEQ ID NO: 134 - DNA, 22 bp, Artificial
  Alu#67 Yb8NBC441 3′Primer
tctcagcctagaccaataccaa

SEQ ID NO: 135 - DNA, 25 bp, Artificial
  Alu#68 Yb8NBC450 5′Primer
tgaaatctatctcgtaggaaggcta

SEQ ID NO: 136 - DNA, 20 bp, Artificial
  Alu#68 Yb8NBC450 3′Primer
ccgctggttaccaaaagatt

SEQ ID NO: 137 - DNA, 22 bp, Artificial
  Alu#69 Yb8NBC461 5′Primer
ccaaagtcattcttcattctgc

SEQ ID NO: 138 - DNA, 23 bp, Artificial
  Alu#69 Yb8NBC461 3′Primer
gacacccgaaaagactaaagaca

SEQ ID NO: 139 - DNA, 20 bp, Artificial
  Alu#70 Yb8NBC463 5′Primer
gccagtgcttgggttttaga

SEQ ID NO: 140 - DNA, 20 bp, Artificial
  Alu#70 Yb8NBC463 3′Primer
ctggcaatgaatttcccttt

SEQ ID NO: 141 - DNA, 25 bp, Artificial
  Alu#71 Yb8NBC466 5′Primer
ttgaggcactagacttacagaattg

SEQ ID NO: 142 - DNA, 20 bp, Artificial
  Alu#71 Yb8NBC466 3′Primer
caggagctgctttcacctct

SEQ ID NO: 143 - DNA, 21 bp, Artificial
  Alu#72 Yb8NBC479 5′Primer
catcctgtttcaacatcagca

SEQ ID NO: 144 - DNA, 20 bp, Artificial
  Alu#72 Yb8NBC479 3′Primer
gttcccagcaggaatctgag

SEQ ID NO: 145 - DNA, 21 bp, Artificial
  Alu#73 Yb8NBC480 5′Primer
cctctctcacaaacagtgcag

SEQ ID NO: 146 - DNA, 20 bp, Artificial
  Alu#73 Yb8NBC480 3′Primer
tcgcaagacacaggctatca

SEQ ID NO: 147 - DNA, 21 bp, Artificial
  Alu#74 Yb8NBC485 5′Primer
tgttcttgccagaaagtttgc

SEQ ID NO: 148 - DNA, 20 bp, Artificial
  Alu#74 Yb8NBC485 3′Primer
ccaatccaggactcgacatt

SEQ ID NO: 149 - DNA, 20 bp, Artificial
  Alu#75 Yb8NBC49 5′Primer
gcagtggattggtttttctg

SEQ ID NO: 150 - DNA, 21 bp, Artificial
  Alu#75 Yb8NBC49 3′Primer
gctgaaagaggcattgaaatc

SEQ ID NO: 151 - DNA, 20 bp, Artificial
  Alu#76 Yb8NBC5 5′Primer
aaggtctaagcgcagtggaa

SEQ ID NO: 152 - DNA, 20 bp, Artificial
  Alu#76 Yb8NBC5 3′Primer
tgtatgcaggttgcttgctc

SEQ ID NO: 153 - DNA, 21 bp, Artificial
  Alu#77 Yb8NBC505 5′Primer
tgagcctatgactgagcatga

SEQ ID NO: 154 - DNA, 20 bp, Artificial
  Alu#77 Yb8NBC505 3′Primer
ggggctctcatcagcattta

SEQ ID NO: 155 - DNA, 21 bp, Artificial
  Alu#78 Yb8NBC516 5′Primer
gggctcagggatactatgctc

SEQ ID NO: 156 - DNA, 20 bp, Artificial
  Alu#78 Yb8NBC516 3′Primer
gcctaggcctaccactcaga

SEQ ID NO: 157 - DNA, 20 bp, Artificial
  Alu#79 Yb8NBC547 5′Primer
gcccatgctcagtctaaacc

SEQ ID NO: 158 - DNA, 20 bp, Artificial
  Alu#79 Yb8NBC547 3′Primer
gattggagcccttgtctacg

SEQ ID NO: 159 - DNA, 20 bp, Artificial
  Alu#80 Yb8NBC568 5′Primer
aaacccaacaaatgtgcttc

SEQ ID NO: 160 - DNA, 21 bp, Artificial
  Alu#80 Yb8NBC568 3′Primer
ggcaacctacacaaagcatgt

SEQ ID NO: 161 - DNA, 21 bp, Artificial
  Alu#81 Yb8NBC576 5′Primer
gggaactaactagtgggcaaa

SEQ ID NO: 162 - DNA, 25 bp, Artificial
  Alu#81 Yb8NBC576 3′Primer
gcatgtacactaaggtatgcaaaag

SEQ ID NO: 163 - DNA, 22 bp, Artificial
  Alu#82 Yb8NBC585 5′Primer
cattgggtttaacattcgctct

SEQ ID NO: 164 - DNA, 20 bp, Artificial
  Alu#82 Yb8NBC585 3′Primer
cacgtgtgcagcaatgtatg

SEQ ID NO: 165 - DNA, 20 bp, Artificial
  Alu#83 Yb8NBC589 5′Primer
agtcttaatgggcgctgaga

SEQ ID NO: 166 - DNA, 20 bp, Artificial
  Alu#83 Yb8NBC589 3′Primer
agtgcctcacccagtagcac

SEQ ID NO: 167 - DNA, 20 bp, Artificial
  Alu#84 Yb8NBC596 5′Primer
tccagggccaagtagtgaat

SEQ ID NO: 168 - DNA, 20 bp, Artificial
  Alu#84 Yb8NBC596 3′Primer
ctgccccaaatgcttacact

SEQ ID NO: 169 - DNA, 20 bp, Artificial
  Alu#85 Yb8NBC597 5′Primer
tgaggtgttgcagacgatgt

SEQ ID NO: 170 - DNA, 21 bp, Artificial
  Alu#85 Yb8NBC597 3′Primer
cgcatgctttagagaataccc

SEQ ID NO: 171 - DNA, 21 bp, Artificial
  Alu#86 Yb8NBC598 5′Primer
tgggtcctatcatcccctatc

SEQ ID NO: 172 - DNA, 20 bp, Artificial
  Alu#86 Yb8NBC598 3′Primer
ccagaaggcatctcatggtt

SEQ ID NO: 173 - DNA, 20 bp, Artificial
  Alu#87 Yb8NBC605 5′Primer
gcctctaggtggagcccttt

SEQ ID NO: 174 - DNA, 20 bp, Artificial
  Alu#87 Yb8NBC605 3′Primer
gccccatttatggctgttta

SEQ ID NO: 175 - DNA, 20 bp, Artificial
  Alu#88 Yb8NBC622 5′Primer
tcaaaacttgcggattttcc

SEQ ID NO: 176 - DNA, 21 bp, Artificial
  Alu#88 Yb8NBC622 3′Primer
tgctgagctatactggtgcaa

SEQ ID NO: 177 - DNA, 20 bp, Artificial
  Alu#89 Yb8NBC636 5′Primer
cctctggcaagctgcttaat

SEQ ID NO: 178 - DNA, 23 bp, Artificial
  Alu#89 Yb8NBC636 3′Primer
tcacagctagaggagacatgaaa

SEQ ID NO: 179 - DNA, 20 bp, Artificial
  Alu#90 Yb8NBC65 5′Primer
atctcatctccctgcctctg

SEQ ID NO: 180 - DNA, 20 bp, Artificial
  Alu#90 Yb8NBC65 3′Primer
gggaggtctggagatctgtg

SEQ ID NO: 181 - DNA, 21 bp, Artificial
  Alu#91 Yb8NBC77 5′Primer
cggaatgttctgaggatcaaa

SEQ ID NO: 182 - DNA, 21 bp, Artificial
  Alu#91 Yb8NBC77 3′Primer
ggaagctctgcacaactccta

SEQ ID NO: 183 - DNA, 20 bp, Artificial
  Alu#92 Yb8NBC80 5′Primer
atttcacagtgccctgtcct

SEQ ID NO: 184 - DNA, 20 bp, Artificial
  Alu#92 Yb8NBC80 3′Primer
tccaggcagatgaattgaca

SEQ ID NO: 185 - DNA, 21 bp, Artificial
  Alu#93 Yb8NBC93 5′Primer
aagtgagtcccagggccttct

SEQ ID NO: 186 - DNA, 20 bp, Artificial
  Alu#93 Yb8NBC93 3′Primer
cacacaggcacttgtttggt

SEQ ID NO: 187 - DNA, 23 bp, Artificial
  Alu#94 Yb9NBC10 5′Primer
gttttcctggtgtgccctaaata

SEQ ID NO: 188 - DNA, 25 bp, Artificial
  Alu#94 Yb9NBC10 3′Primer
tttacctaactcacaagacccaaag

SEQ ID NO: 189 - DNA, 25 bp, Artificial
  Alu#95 Yb9NBC50 5′Primer
gttccacaagtacaggagaaaatgt

SEQ ID NO: 190 - DNA, 25 bp, Artificial
  Alu#95 Yb9NBC50 3′Primer
gaagctctttaggaaaccaaatctc

SEQ ID NO: 191 - DNA, 23 bp, Artificial
  Alu#96 Yc1NBC2 5′Primer
tctctcatgaacatagatacaaa

SEQ ID NO: 192 - DNA, 20 bp, Artificial
  Alu#96 Yc1NBC2 3′Primer
cgtgcattcttgagataaat

SEQ ID NO: 193 - DNA, 20 bp, Artificial
  Alu#97 Yc1NBC35 5′Primer
cccattctccatgccgtgat

SEQ ID NO: 194 - DNA, 20 bp, Artificial
  Alu#97 Yc1NBC35 3′Primer
tgcaaggcattggggataca

SEQ ID NO: 195 - DNA, 22 bp, Artificial
  Alu#98 Yc1NBC53 5′Primer
aaagctatcaaccatgccaaca

SEQ ID NO: 196 - DNA, 22 bp, Artificial
  Alu#98 Yc1NBC53 3′Primer
gaaaatgctattttggggaatg

SEQ ID NO: 197 - DNA, 22 bp, Artificial
  Alu#99 Yc1NBC63 5′Primer
ggtactcagtaacacatcaaga

SEQ ID NO: 198 - DNA, 20 bp, Artificial
  Alu#99 Yc1NBC63 3′Primer
aagctgggtggtgggttcac

SEQ ID NO: 199 - DNA, 22 bp, Artificial
  Alu#100 Yc1RG68 5′Primer
atggtgtccacaagaaactgag

SEQ ID NO: 200 - DNA, 23 bp, Artificial
  Alu#100 Yc1RG68 3′Primer
ggaaggctccattataggtcttg

(19) United States
(12) Patent Application Publication
(10) Pub. No.: US 2007/0003944 A1
(43) Pub. Date: Jan. 4, 2007

(54) Inference of human geographic origins using Alu insertion polymorphisms

(76) Inventors: Sudhir K. Sinha, New Orleans, LA (US); Jaiprakash G. Shewale, New Orleans, LA (US); Mark A. Batzer, Mandeville, LA (US); Jerilyn A. Walker, Breaux Bridge, LA (US); David A. Ray, Morgantown, WV (US)

Correspondence Address: Robert E. Bushnell;Suite 300, 1522 K Street, N.W., Washington, DC, 20005, US

(21) Appl. No.: 11/202,247

(22) Filed: Aug. 12, 2005

Related U.S. Application Data
(60) Provisional application No. 60/635,441, filed on Dec. 14, 2004.

(51) Int. Cl. C12Q 1/68; C12P 19/34

(52) U.S. Cl. 435/6; 435/91.2

(57) ABSTRACT

The insertion polymorphisms based on interspersed elements including LINEs and SINEs is used for the inference of an individual's geographic origin. SINE polymorphisms are identical-by-descent, essentially homoplasy-free, and inexpensive to genotype using a variety of approaches. Using a Structure analysis of the Alu insertion polymorphism based genotypes, the geographic affiliation of unknown human individuals can be inferred with high levels of confidence. This technique to infer the geographic affiliation of unknown human DNA samples can be a useful tool in forensic genomics.

### CLAIM OF PRIORITY

This application makes reference to, incorporates the same herein, and claims all benefits accruing under 35 U.S.C. §119 from a provisional application for INFERENCE OF HUMAN GEOGRAPHIC ORIGINS USING ALU INSERTION POLYMORPHISMS earlier filed in the United States Patent & Trademark Office on 14 Dec. 2004 and there duly assigned Ser. No. 60/635,441.

### GOVERNMENT SUPPORT

This invention was supported by award N41756-03-C-4063 from the Technical Support Working Group (M.A.B.).

### BACKGROUND OF INVENTION

1. Field of Invention

The present invention relates to inference of human geographic origins using Alu insertion polymorphisms.

2. Description of the Related Art

Forensic DNA specimens are routinely matched to alleged criminal suspects in modern law enforcement. Frequently however, tools that narrow the potential pool of suspects are essential precursors to a positive identification in investigative forensics. The inferred ancestral origin of a DNA specimen is one type of evidence that can aid a criminal investigation. Human genetic variation and geographic population affiliation have been studied using many genetic systems, including mitochondrial (see M. Bamshad et al., Genome Res. 11 (2001) 994-1004; L. B. Jorde et al., Am. J. Hum. Genet. 66 (2000) 979-988; B. Budowle et al., Forensic Sci. Int. 103 (1999) 23-35), Y-chromosome (see M. Bamshad et al., Genome Res. 11 (2001) 994-1004; L. B. Jorde et al., Am. J. Hum. Genet. 66 (2000) 979-988), microsatellite (see M. J. Bamshad et al., Am. J. Hum. Genet. 72 (2003) 578-589; L. B. Jorde et al., Proc. Natl. Acad. Sci. U.S.A. 94 (1997) 3100-3103), short tandem repeats (STR) (see L. B. Jorde et al., Am. J. Hum. Genet. 66 (2000) 979-988; J. M. Butler et al., J. Forensic Sci. 48 (2003) 908-911; B. Budowle et al., J. Forensic Sci. 46 (2001) 453-489; M. D. Shriver et al., Am. J. Hum. Genet. 60 (1997) 957-964), mobile elements (see M. J. Bamshad et al., Am. J. Hum. Genet. 72 (2003) 578-589; M. A. Batzer et al., J. Mol. Evol. 42 (1996) 22-29; M. Stoneking et al., Genome Res. 7 (1997) 1061-1071; C. Romualdi et al., Genome Res. 12 (2002) 602-612; W. S. Watkins et al., Genome Res. 13 (2003) 1607-1618; W. S. Watkins et al., Am. J. Hum. Genet. 68 (2001) 738-752; A. M. Roy-Engel et al., Genetics 159 (2001) 279-290), and single nucleotide polymorphisms (SNPs) (see R. Sachidanandam, et al., Nature 409 (2001) 928-933; T. C. Matise et al., Am. J. Hum. Genet. 73 (2003) 271-284; D. E. Reich et al., Nat. Genet. 33 (2003) 457-458; B. A. Salisbury et al., Mutat. Res. 526 (2003) 53-61.)

Recently, Frudakis, et al. developed a SNP-based system for inference of ancestry for application to forensic casework. (See T. Frudakis et al., J. Forensic Sci. 48 (2003) 771-782.) The initial system consisted of 56 SNP loci targeted from pigmentation and xenobiotic metabolism genes with ancestral diversity designed to identify individuals of European, African, and Asian descent. (See T. Frudakis et al., J. Forensic Sci. 48 (2003) 771-782.) Subsequently, Frudakis and DNAPrint™ Genomics, Inc. (Sarasota, Fla.) have introduced commercial applications of various SNP-based systems as a forensic service to law enforcement agencies. Notably, DNAWITNESS™ 2.0 was instrumental for inferring the geographic origin of the Louisiana serial killer in 2003 (www.dnaprint.com).

Although emerging SNP-based technologies have recently proven quite useful in law enforcement and will undoubtedly remain so in the future, SNPs have some limitations due the fact that they represent single base pair differences. Like most other genetic polymorphisms, SNPs can be merely identical-by-state; that is, they may have arisen as a result of an independent parallel forward or backward mutation resulting in genotype misclassification (homoplasy).

### SUMMARY OF THE INVENTION

It is therefore an object of the present invention to provide a process for determining the human geographic origin of an unknown human DNA sample.

It is another object of the present invention to provide a primer adapted for determining the human geographic origin of an unknown human DNA sample.

We introduce the use of insertion polymorphisms based on interspersed elements including long interspersed elements (LINEs) and short interspersed elements (SINEs) as an alternative to existing systems. Mobile element insertion polymorphisms are essentially homoplasy-free characters, identical by descent (see E. S. Lander, et al., Nature 409 (2001) 860-921; M. A. Batzer and P. L. Deininger, Nat. Rev. Genet. 3 (2002) 370-379; B. J. Vincent et al., Mol. Biol. Evol. 20 (2003) 1338-1348), and easy to genotype in a variety of formats (see M. J. Bamshad et al., Am. J. Hum. Genet. 72 (2003) 578-589; P. A. Callinan et al., Gene 317 (2003) 103-110; M. L. Carroll et al., J. Mol. Biol. 311 (2001) 17-40; D. J. Hedges et al., Anal. Biochem. 312 (2003) 77-79); D. H. Kass et al., Anal. Biochem. 321 (2003) 146-149). The ancestral state of a human mobile element insertion polymorphism is known to be the absence of the element at a particular genomic location (see M. Stoneking et al., Genome Res. 7 (1997) 1061-1071). Alu elements are approximately 300 nucleotides in length and represent the most abundant class of short interspersed mobile elements (SINEs) in the human genome with more than one million copies (see E. S. Lander, et al., Nature 409 (2001) 860-921). Most of these elements are “fixed”, meaning that all individuals are homozygous for the insertion at a particular locus. However, members of several young Alu subfamilies such as Ya5, Ya8, Yb8, Yb9, Yc1, Yc2 and others, are polymorphic for insertion presence/absence (see M. A. Batzer et al., Nat. Rev. Genet. 3 (2002) 370-379; A. B. Carter et al., Hum. Gen. 1 (2004) 167-178; A. C. Otieno et al., Analysis of the Human Alu Ya-Lineage, J. Mol. Biol. 342 (2004) 109-118) and different numbers of such markers have been shown to provide robust measurements of the relationships among various world populations. (See M. Stoneking et al., Genome Res. 7 (1997) 1061-1071; W. S. Watkins et al., Genome Res. 13 (2003) 1607-1618; W. S. Watkins et al., Am. J. Hum. Genet. 68 (2001) 738-752; M. A. Batzer et al., Proc. Natl. Acad. Sci. U.S.A. 91 (1994) 12288-12292.) These features make mobile element insertion polymorphisms virtual genomic fossils of ancestral lineage and thus a valuable tool for determining human geographic origins.

Here, we report the application of 100 Alu insertion polymorphisms as a forensic tool to ascertain the inferred geographic origin of unknown human DNA samples. In this blind study, we examined DNA specimens from 18 geographically diverse humans. For each sample, we used multi-locus genotypes from Alu insertion polymorphisms to infer geographic affiliation from among four major world populations.

The present invention may be constructed with a process for determining the human geographic origin of an unknown human DNA sample, the process including determining the human geographic origin of the DNA sample using insertion polymorphisms based on interspersed elements.

According to another aspect of the present invention, a process for determining an ancestry of an unknown DNA sample, including: amplifying Alu elements in the unknown DNA sample, said Alu elements being polymorphic for insertion presence/absence; deriving a genotype for the unknown sample from the amplified Alu elements; and determining the human geographic origin of the unknown DNA sample by calculating the frequency of the genotype from a reference database.

The inference of an individual's geographic origin can be critical in narrowing the field of potential suspects in a criminal investigation. Most current technologies rely on single nucleotide polymorphism (SNP) genotypes to accomplish this task. However, SNPs can introduce homoplasy into an analysis since they can be identical-by-state. We introduce the use of insertion polymorphisms based on short interspersed elements (SINEs) as an alternative to SNPs. SINE polymorphisms are identical-by-descent, essentially homoplasy-free, and inexpensive to genotype using a variety of approaches. Herein, we present results of a blind study using 100 Alu insertion polymorphisms to infer the geographic ancestry of 18 unknown individuals from a variety of geographic locations. Using a Structure analysis of the Alu insertion polymorphism based genotypes, we were able to correctly infer the geographic affiliation of all 18 unknown human individuals with high levels of confidence. This technique to infer the geographic affiliation of unknown human DNA samples can be a useful tool in forensic genomics.

### BRIEF DESCRIPTION OF THE DRAWING

A more complete appreciation of the present invention, and many of the above and other features and advantages of the present invention, will be readily apparent as the same becomes better understood by reference to the following detailed description when considered in conjunction with the accompanying drawings in which like reference symbols indicate the same or similar components, wherein:

FIGS. 1-1 through 1-3 shows a table listing the Alu elements oligonucleotide primers and amplification conditions;

FIG. 2 illustrates an example of gel electrophoresis results for the 18 individuals at three Alu insertion loci; and

FIG. 3 shows genotype data for 18 unknown DNA samples for nine of the 100 Alu loci used.

### DETAILED DESCRIPTION OF THE PREFERRED EMBODIMENTS

Materials and Methods

DNA Samples

Eighteen anonymous human DNA samples were obtained under informed consent for this experiment by the Illinois State Police Forensic Science Center at Chicago and the National Center for Forensic Science, University of Central Florida in Orlando. The DNA from each sample was extracted from bloodstain cards or buccal swabs by the source laboratories (Illinois State Police and National Center for Forensic Science) and shipped to Louisiana State University (LSU) for genetic analysis using 100 Alu insertion polymorphisms and a mobile element based sex typing assay (see D. J. Hedges, J. A. Walker, P. A. Callinan, J. G. Shewale, S. K. Sinha and M. A. Batzer, Mobile Element-Based Assay for Human Gender Determination, Anal. Biochem. 312 (2003) 77-79 which is incorporated herein by reference). Investigators from each source laboratory had access to the physical description and geographic ancestry of the anonymous subjects while the analysis team at LSU remained blind to this data until the conclusion of the study.

Alu Elements and PCR Amplification

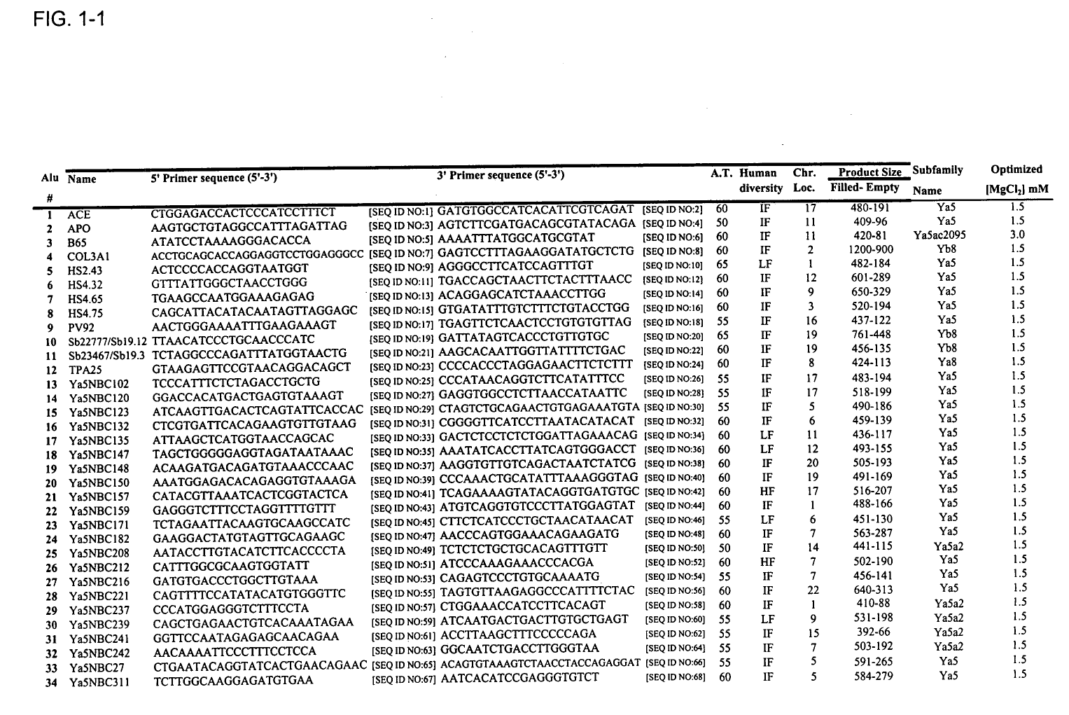

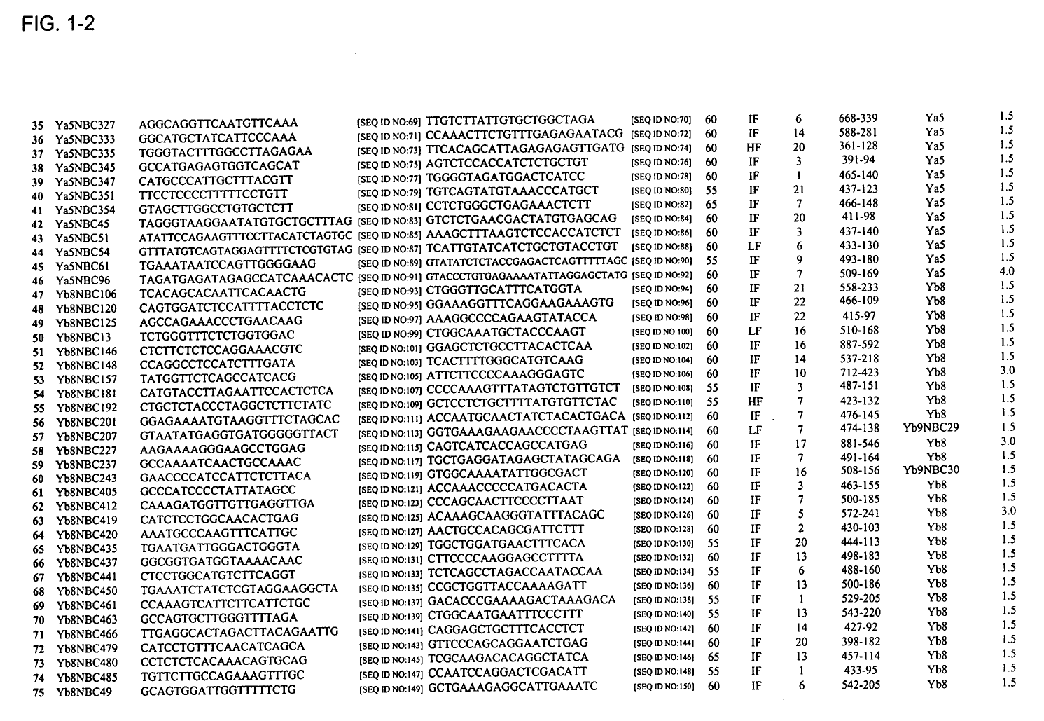

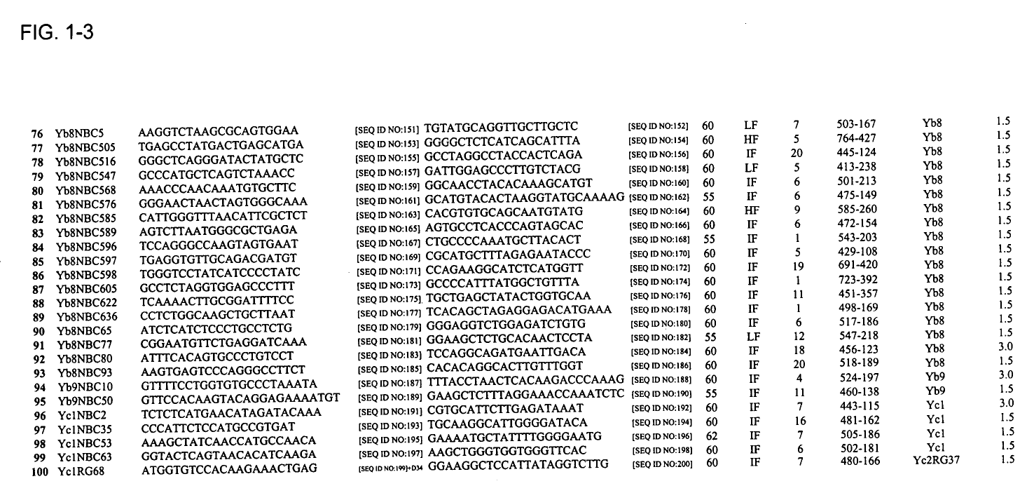

One hundred Alu insertion polymorphisms were used in this study. A complete list of the Alu elements oligonucleotide primers and amplification conditions is shown in FIGS. 1-1 through 1-3. In FIGS. 1-1 through 1-3, A.T. is the annealing temperature used in each PCR reaction, and human diversity (H.D.) for each polymorphic Alu is listed as LF (low frequency), IF (intermediate frequency), or HF (high frequency). It is also available at the website (http://batzerlab.lsu.edu) and at http://www.genome.org as supplemental material for Watkins et al. (see M. J. Bamshad, S. Wooding, W. S. Watkins, C. T. Ostler, M. A. Batzer and L. B. Jorde, Human Population Genetic Structure and Inference of Group Membership, Am. J. Hum. Genet. 72 (2003) 578-589.

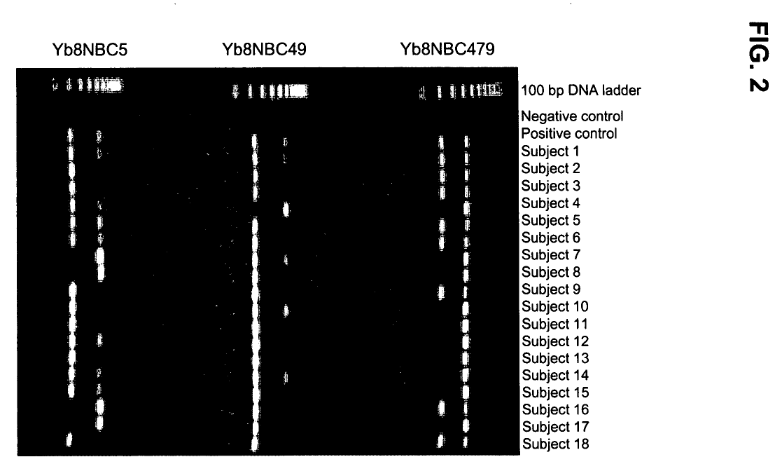

PCR reactions for agarose gel based detection were carried out in 25 μl using 10 ng of DNA template, 1×PCR buffer II (Applied Biosystems, Inc.), 0.2 mM dNTPs, 200 nM each oligonucleotide primer, optimized MgCl₂, and one unit Taq DNA polymerase. Each sample was subjected to an initial denaturation of one minute at 95° C. followed by 32 amplification cycles of denaturation at 95° C. for 30 seconds, optimized annealing for 30 seconds, followed by extension at 72° C. for 30 seconds. Amplicons were size-separated on a 2% agarose gel containing 0.2 μg/ml ethidium bromide and visualized by UV illumination (FIG. 2). Human gender identification was performed using sex chromosome specific mobile elements as previously reported by Hedges et al. (see D. J. Hedges, J. A. Walker, P. A. Callinan, J. G. Shewale, S. K. Sinha and M. A. Batzer, Mobile Element-Based Assay for Human Gender Determination, Anal. Biochem. 312 (2003) 77-79 which is incorporated herein by reference).

Data Analysis and Structure Inference

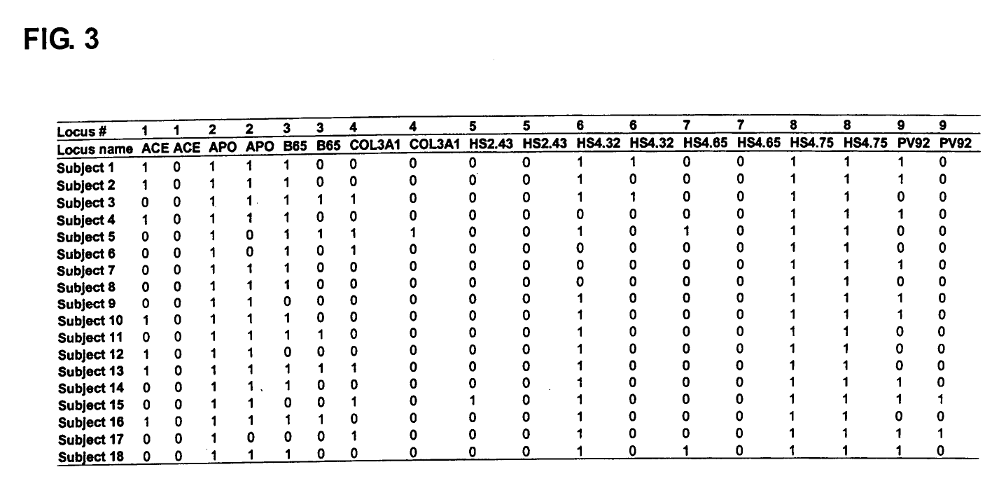

Genotypic data were recorded for each allele as follows: an individual who was homozygous present for a given Alu locus was assigned the code 1, 1; homozygous absent, 0, 0; and heterozygous, 1, 0. A sample of the data is shown in FIG. 3, wherein the genotype data for 18 unknown DNA samples for nine of the 100 Alu loci used is shown. For each locus, there are two entries indicating the genotype of the sample. “1” indicates the presence of the Alu element at that allele and “0” indicates the absence of the element. The complete reference database is available at the website, http://batzerlab.lsu.edu under publication “Inference of human geographic origins using Alu insertion polymorphisms,” Forensic Science International (In press), as an electronic appendix., which is incorporated herein by reference.

The geographic affiliation of the samples was inferred using Structure 2.0. The Structure program is described in D. Falush, M. Stephens and J. K. Pritchard, Genetics 164 (2003) 1567-1587, N. A. Rosenberg, L. M. Li, R. Ward and J. K. Pritchard, Am. J. Hum. Genet. 73 (2003) 1402-1422, and J. K. Pritchard, M. Stephens and P. Donnelly, Genetics 155 (2000) 945-959, which are incorporated herein by reference. This software package performs model-based clustering using genotypic data from unlinked markers to infer population structure. For each individual, Structure 2.0 estimates the proportion of ancestry from each of K clusters. We used a burn-in of 15,000 iterations and a run of 20,000 replications. The sample size was 715 individuals of known geographic ancestry, plus eighteen individuals of unknown ancestry for a total of 733. Because previous analyses of the same known data indicated the presence of four distinct populations (see M. J. Bamshad, S. Wooding, W. S. Watkins, C. T. Ostler, M. A. Batzer and L. B. Jorde, Human population genetic structure and inference of group membership. Am. J. Hum. Genet. 72 (2003) 578-589), the expected number of populations (K) was set at four (European, African, Asian, or Indian). Three replicate runs were performed on the dataset, each requiring about 20 minutes using a desktop computer with a 3 GHz processor.

Results

In our analysis of the eighteen anonymous DNA samples, the amplification efficiency at each of the 100 Alu loci was 100%. Population assignment probabilities obtained from Structure 2.0 using the genotype data are outlined in Table 1.

TABLE 1
Probabilities of population origin for 18 unknown human DNA samples
inferred using Structure 2.0. Values used to assign geographic
affiliation are shown in bold. Gender (G) is shown as female (F)
or male (M) and matches data from the source laboratories.

| | | | | | | | Actual |
|---|---|---|---|---|---|---|---|
| | | | | | | | Population |
| | | | | | | | of Origin |
| | Inferred Population Origin | | | | | St. | (revealed |
| Sample ID | G | Africa | Asia | Europe | India | Dev. | post-analysis) |
| Subject 1 | F | 0.002 | 0.034 | 0.892 | 0.072 | 0.026 | European |
| Subject 2 | F | 0.039 | 0.023 | 0.923 | 0.015 | 0.007 | European |
| Subject 3 | M | 0.011 | 0.030 | 0.935 | 0.024 | 0.005 | European |
| Subject 4 | F | 0.004 | 0.016 | 0.977 | 0.004 | 0.001 | European |
| Subject 5 | F | 0.847 | 0.026 | 0.062 | 0.065 | 0.011 | African- |
| | | | | | | | American |
| Subject 6 | F | 0.647 | 0.033 | 0.224 | 0.096 | 0.008 | African- |
| | | | | | | | American |
| Subject 7 | F | 0.010 | 0.011 | 0.973 | 0.006 | 0.004 | European |
| Subject 8 | M | 0.003 | 0.009 | 0.978 | 0.010 | 0.001 | European |
| Subject 9 | F | 0.252 | 0.010 | 0.715 | 0.022 | 0.005 | Jamaican |
| Subject 10 | F | 0.003 | 0.005 | 0.964 | 0.028 | 0.008 | Greece |
| Subject 11 | F | 0.005 | 0.013 | 0.937 | 0.046 | 0.009 | Finland |
| Subject 12 | F | 0.015 | 0.032 | 0.923 | 0.030 | 0.003 | England |
| Subject 13 | M | 0.003 | 0.002 | 0.991 | 0.004 | 0.001 | Scotland |
| Subject 14 | F | 0.008 | 0.006 | 0.981 | 0.005 | 0.002 | Italy |
| Subject 15 | M | 0.002 | 0.100 | 0.864 | 0.034 | 0.028 | Venezuela |
| Subject 16 | M | 0.511 | 0.056 | 0.383 | 0.050 | 0.011 | African- |
| | | | | | | | American |
| Subject 17 | M | 0.010 | 0.459 | 0.040 | 0.491 | 0.043 | India |
| Subject 18 | M | 0.005 | 0.938 | 0.044 | 0.013 | 0.009 | Chinese |

Of the 18 unknown samples, 14 were assigned to one population with a probability greater than 80% (N=12 were identified as European, N=1 was identified as African/African-American, and N=1 was identified as Asian). The remaining 4 samples were classified as being of mixed ancestry (N=3 an admixture of European and African descent; and N=1 an admixture of Indian and Asian descent). Information revealed by the source laboratories following the study listed DNA samples #1-4, #7, #8 as European, and #5-6, #16 as African American. DNA sample #9 was listed as Jamaican, #10 of Greek ancestry, #11 as from Finland, #12 from England, #13 from Scotland, #14 from Italy, #15 from Venezuela, #17 from India, and #18 as Chinese. Our results for samples #10-14 suggested that these were European in origin with a 92-99% probability. Sample #18 was identified as being of Asian descent with a 94% probability. Sample #15 tested as an admixture of 86% European/10% Asian, which is consistent with a Venezuelan origin.

The four samples classified as having mixed geographic origin (<80% identity with one of the primary populations) were subjected to secondary analyses to obtain detailed admixture information. Based on Structure's estimate of the most likely population(s) of origin, samples were assigned to each of the two potential source populations and admixture estimates were calculated for three parental generations. When samples #6 and #16 were assigned to Africa, the admixture analyses showed weak agreement that both were exclusively African (30% and 27%, respectively) with a 16-23% likelihood that at least one parent or grandparent was of European ancestry. Conversely, when #6 and #16 were assigned to the European population, there were strong indications of genetic contributions from an African parent for each subject, 99% and 76%, respectively. Both individuals were confirmed as African-American by the source laboratories. When sample #9 (Jamaican) was assigned to the European population, admixture analyses indicated a <1% likelihood that this was true, and a 47% probability that at least one grandparent or great-grandparent was of African descent. Conversely, when #9 was assigned to the African population, admixture analyses indicated a 6% likelihood that this was true, and an 87% probability that at least one parent was of European ancestry. Subject #17 (identified as from India by the source laboratory) showed the most admixture of the eighteen unknowns tested with strong affinity for both Indian (95%) and Asian (85%) populations, as well as a 24% probability that at least one great-grandparent was of European ancestry.

Variation in probability of assignment between the three original runs ranged from 0.1% to 7.9% (data not shown), with most (15/18) samples having a standard deviation of less than 0.012. The inferred geographic affiliation was consistent for all samples across the three runs. The standard deviation of population probability assignments among runs (average st. dev.=0.10) is shown for each sample in Table 1. The raw output for each of the three original runs and the secondary runs for detecting admixture levels is available on the webpage, http://batzerlab.lsu.edu under publication “Inference of human geographic origins using Alu insertion polymorphisms,” Forensic Science International (In Press), as an electronic appendix, 3-Structure Output, which is incorporated herein by reference.

The results of our study demonstrate the utility of this approach as a forensic tool. Determining the human geographic origin of an unknown human DNA sample could aid a criminal investigation by narrowing the pool of potential suspects. The Markov Chain Monte Carlo methodology used by the Structure 2.0 software package provides a powerful analysis to group all individuals into the selected number of populations and then determine the probability that each individual belongs to any given group. In addition, the software has the ability to detect admixture between populations in individual genotypes going back several parental generations. We were successful in determining the geographic origin of the 18 unknown human DNA samples. Many of the probabilities of assignment were well over 80% and the detection of admixture in individuals of mixed ancestry was easily identified. Only one sample, #17 (see Table 1), gave results that might be considered ambiguous. However, given the complicated makeup of the Indian population, this result is not unexpected. Indeed, of the four populations in the current database of Alu insertion polymorphism variation, India is by far the most heterogeneous with many individuals clustering with either Europe or East Asia (see M. J. Bamshad, S. Wooding, W. S. Watkins, C. T. Ostler, M. A. Batzer and L. B. Jorde, Human population genetic structure and inference of group membership. Am. J. Hum. Genet. 72 (2003) 578-589). The results of our analyses were also consistent between runs suggesting that, in practice within investigative forensic laboratories, single runs of the analysis are all that would be sufficient.

The 100 Alu insertion polymorphisms used in this study were largely mined from existing human genome databases. However, since the human dispersal from Africa, Alu elements have continued to expand in the human genome. For example, the more recent the insertion, the more likely it is to occur at high frequency in the geographic region of origin and exhibit very low alleles frequencies elsewhere, thus being indicative of its specific source population. The incorporation of additional population-indicative mobile element insertion polymorphisms to the existing panel of markers will eventually allow for subgroup (sub-continental) affiliation tests. We are in the process of implementing a cascade-like strategy to our method, which will consist of a series of tiered analyses for determination of “primary” geographic affiliation (Africa, Europe, Asia, or India), then for “secondary” or subgroup affiliation within each of these broad continental groups. Thus, once the initial Structure 2.0 analysis narrows the sample origin to a continental affiliation, subsequent analyses, using only insertion loci that are useful within one of these continental populations, have the potential to further isolate the unknown sample to sub-continental and regional origin. We are currently identifying additional mobile element insertion polymorphisms using PCR based displays and data mining to identify sub-continental patterns of variation.

Previously, one limitation to this type of multiple locus approach has been that forensic DNA samples are often only available in trace quantities. The analysis of 100 separate PCR amplicons requires significantly more than trace amounts. Recent advancements in whole genome amplification (WGA) technologies such as RepliPHI™ (EPICENTRE, Madison, Wis.) and GenomiPhi™ (Amersham Biosciences, Newark, N.J.) have virtually eliminated this obstacle. Genomic DNA from residual cells left by incidental contact can be subjected to WGA and produce amplification patterns from the WGA templates which are completely consistent with the patterns observed using the original genomic DNA (see K. J. Sorensen, K. Turteltaub, G. Vrankovich, J. Williams and A. T. Christian, Whole-genome amplification of DNA from residual cells left by incidental contact. Anal. Biochem. 324 (2004) 312-314, which is incorporated herein by reference).

In an effort to confirm this for the 100 Alu insertion polymorphisms, we recently compared amplification patterns using original genomic DNA and WGA DNA. An aliquot of the original DNA was sent from LSU to LLNL where it was WGA using the method of Sorensen et al. (see K. J. Sorensen, K. Turteltaub, G. Vrankovich, J. Williams and A. T. Christian, Whole-genome amplification of DNA from residual cells left by incidental contact. Anal. Biochem. 324 (2004) 312-314, which is incorporated herein by reference) and then returned to LSU for comparative analyses. The genotypes were 97% (473 out of 489) consistent between the original DNA and the WGA DNA. Each of the 16 (of 489) disagreements (3%) represented a single allele aberration (i.e. between heterozygous and homozygous). The ability to determine the inferred geographic origin of each individual was unaffected and was 100% consistent between the original and WGA DNA. The complete genotype results of this WGA experiment are presented on the website, http://batzerlab.lsu.edu under publication “Inference of human geographic origins using Alu insertion polymorphisms,” Forensic Science International (In Press), as an electronic appendix, 4-WGA results, which is incorporated herein by reference.

There are several advantages to the use of Alu insertion polymorphisms for the inference of human geographic origins. First, it can be a “low-tech” approach using standard PCR thermal cyclers and simple agarose gel electrophoresis commonly available in most laboratories. Second, Alu insertions are about 300 nucleotides long, identical by descent, and thus quite stable compared to single nucleotide differences subject to forward or backward mutations. Furthermore, as more recent and more population-indicative Alu insertions are discovered and integrated into the analyses, the number of elements required to meet the needs of the investigator will decrease.

In most routine criminal investigations, inference of geographic origin may be defined simply as Caucasian, African-American, or Asian, making our 100 Alu approach seem excessive. However, as law enforcement becomes increasingly global, the powerful statistical capability of our Alu-based approach using Structure will likely prove useful. While the analysis of the Alu genotype data can be accomplished relatively quickly (<20 minutes on a 3 Ghz processor), the development of multiplex compatible systems will be useful for the transition of this approach to the forensic community. Although, multiplex PCR has been successful in testing 3 to 4 Alu element insertions simultaneously, at least 25 separate PCR reactions would still be required for data collection using these manual systems. Therefore, more automated multiplexed genetic systems using high throughput analysis technology are currently under development. These involve fixing genomic DNA sequences representative of the “Alu present” sites and the pre-integration sites for the 100 Alu insertion polymorphisms such that DNA from an unknown individual can be screened using micro-plate or micro-array based techniques.

Although there are pros and cons to every approach, the inference of an individual's geographic origin is undoubtedly a useful bit of information when trying to narrow the pool of potential suspects during a criminal investigation. Here, we have presented results, which demonstrate that analysis of 100 Alu insertion polymorphisms can be a powerful tool to accurately infer geographic origin. This method can be a useful tool in forensic investigations. Furthermore, the eighteen anonymous human DNA samples used for this experiment were obtained directly from forensic science laboratories (Illinois State Police Forensic Science Center at Chicago and the National Center for Forensic Science, University of Central Florida in Orlando), illustrating the community's interest in this approach.

Although the preferred embodiments of the present invention have been described, those skilled in the art will appreciate that a variety of modifications and changes can be made without departing from the idea and the scope of the present invention described in the following claims.

What is claimed is:

1. A process for determining human geographic origin of an unknown DNA sample using insertion polymorphisms based on interspersed elements.

2. The process of claim 1, wherein the interspersed elements are long interspersed elements (LINEs).

3. The process of claim 1, wherein the interspersed elements are short interspersed elements (SINEs).

4. The process of claim 1, wherein the step of determining the human geographic origin of the DNA sample comprises using multi-locus genotypes from Alu insertion polymorphisms.

5. A process for determining human geographic origin of an unknown DNA sample, comprising the steps of:
extraction of DNA from the unknown biological sample;
amplifying Alu elements in the unknown DNA sample;
obtaining the genotype of unknown sample by detection of amplified products, said Alu elements being polymorphic for insertion presence/absence;
determining the human geographic origin of the unknown DNA sample by calculating the frequency of the genotype from a reference database.

6. The process of claim 5, wherein the step of determining the human geographic origin of the DNA sample further comprises using a model-based clustering method to infer the human geographic origin.

7. The process of claim 5, wherein the amplification of the interspersed elements in the unknown DNA sample comprises carrying out polymerase chain reactions by using oligonucleotide primers that enable detection of Alu elements.

8. The process of claim 1, wherein the human geographic origin is selected from the group which includes African ancestry, Asian ancestry, European ancestry, and Indian ancestry.

9. The process of claim 7, wherein the loci of the Alu elements comprises ACE, APO, B65, COL3A1, HS2.43, HS4.32, HS4.65, HS4.75, and PV92.

10. The process of claim 7, wherein the loci of the Alu elements comprise ACE, APO, B65, COL3A1, HS2.43, HS4.32, HS4.65, HS4.75, PV92, Sb22777/Sb19.12, Sb23467/Sb19.3, TPA25, Ya5NBC102, Ya5NBC120, Ya5NBC123, Ya5NBC132, Ya5NBC135, Ya5NBC147, Ya5NBC148, Ya5NBC150, Ya5NBC157, Ya5NBC159, Ya5NBC171, Ya5NBC182, Ya5NBC208, Ya5NBC212, Ya5NBC216, Ya5NBC221, Ya5NBC237, Ya5NBC239, Ya5NBC241, Ya5NBC242, Ya5NBC27, Ya5NBC311, Ya5NBC327, Ya5NBC333, Ya5NBC335, Ya5NBC345, Ya5NBC347, Ya5NBC351, Ya5NBC354, Ya5NBC45, Ya5NBC51, Ya5NBC54, Ya5NBC61, Ya5NBC96, Yb8NBC106, Yb8NBC120, Yb8NBC125, Yb8NBC13, Yb8NBC146, Yb8NBC148, Yb8NBC157, Yb8NBC181, Yb8NBC192, Yb8NBC201, Yb8NBC207, Yb8NBC227, Yb8NBC237, Yb8NBC243, Yb8NBC405, Yb8NBC412, Yb8NBC419, Yb8NBC420, Yb8NBC435, Yb8NBC437, Yb8NBC441, Yb8NBC450, Yb8NBC461, Yb8NBC463, Yb8NBC466, Yb8NBC479, Yb8NBC480, Yb8NBC485, Yb8NBC49, Yb8NBC5, Yb8NBC505, Yb8NBC516, Yb8NBC547, Yb8NBC568, Yb8NBC576, Yb8NBC585, Yb8NBC589, Yb8NBC596, Yb8NBC597, Yb8NBC598, Yb8NBC605, Yb8NBC622, Yb8NBC636, Yb8NBC65, Yb8NBC77, Yb8NBC80, Yb8NBC93, Yb9NBC10, Yb9NBC50, Yc1NBC2, Yc1NBC35, Yc1NBC53, Yc1NBC63, and Yc1RG68.

11. The process of claim 10, wherein the loci of ACE, APO, B65, COL3A1, HS2.43, HS4.32, HS4.65, HS4.75, PV92, Sb22777/Sb19.12, Sb23467/Sb19.3, TPA25, Ya5NBC102, Ya5NBC120, Ya5NBC123, Ya5NBC132, Ya5NBC135, Ya5NBC147, Ya5NBC148, Ya5NBC150, Ya5NBC157, Ya5NBC159, Ya5NBC171, Ya5NBC182, Ya5NBC208, Ya5NBC212, Ya5NBC216, Ya5NBC221, Ya5NBC237, Ya5NBC239, Ya5NBC241, Ya5NBC242, Ya5NBC27, Ya5NBC311, Ya5NBC327, Ya5NBC333, Ya5NBC335, Ya5NBC345, Ya5NBC347, Ya5NBC351, Ya5NBC354, Ya5NBC45, Ya5NBC51, Ya5NBC54, Ya5NBC61, Ya5NBC96, Yb8NBC106, Yb8NBC120, Yb8NBC125, Yb8NBC13, Yb8NBC146, Yb8NBC148, Yb8NBC157, Yb8NBC181, Yb8NBC192, Yb8NBC201, Yb8NBC207, Yb8NBC227, Yb8NBC237, Yb8NBC243, Yb8NBC405, Yb8NBC412, Yb8NBC419, Yb8NBC420, Yb8NBC435, Yb8NBC437, Yb8NBC441, Yb8NBC450, Yb8NBC461, Yb8NBC463, Yb8NBC466, Yb8NBC479, Yb8NBC480, Yb8NBC485, Yb8NBC49, Yb8NBC5, Yb8NBC505, Yb8NBC516, Yb8NBC547, Yb8NBC568, Yb8NBC576, Yb8NBC585, Yb8NBC589, Yb8NBC596, Yb8NBC597, Yb8NBC598, Yb8NBC605, Yb8NBC622, Yb8NBC636, Yb8NBC65, Yb8NBC77, Yb8NBC80, Yb8NBC93, Yb9NBC10, Yb9NBC50, Yc1NBC2, Yc1NBC35, Yc1NBC53, Yc1NBC63, and Yc1RG68 are amplified by using oligonucleotide primer pairs of SEQ ID NO:1 and SEQ ID NO:2, SEQ ID NO:3 and SEQ ID NO:4, SEQ ID NO:5 and SEQ ID NO:6, SEQ ID NO:7 and SEQ ID NO:8, SEQ ID NO:9 and SEQ ID NO:10, SEQ ID NO:11 and SEQ ID NO:12, SEQ ID NO:13 and SEQ ID NO:14, SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:23 and SEQ ID NO:24, SEQ ID NO:25 and SEQ ID NO:26, SEQ ID NO:27 and SEQ ID NO:28, SEQ ID NO:29 and SEQ ID NO:30, SEQ ID NO:31 and SEQ ID NO:32, SEQ ID NO:33 and SEQ ID NO:34, SEQ ID NO:35 and SEQ ID NO:36, SEQ ID NO:37 and SEQ ID NO:38, SEQ ID NO:39 and SEQ ID NO:40, SEQ ID NO:41 and SEQ ID NO:42, SEQ ID NO:43 and SEQ ID NO:44, SEQ ID NO:45 and SEQ ID NO:46, SEQ ID NO:47 and SEQ ID NO:48, SEQ ID NO:49 and SEQ ID NO:50, SEQ ID NO:51 and SEQ ID NO:52, SEQ ID NO:53 and SEQ ID NO:54, SEQ ID NO:55 and SEQ ID NO:56, SEQ ID NO:57 and SEQ ID NO:58, SEQ ID NO:59 and SEQ ID NO:60, SEQ ID NO:61 and SEQ ID NO:62, SEQ ID NO:63 and SEQ ID NO:64, SEQ ID NO:65 and SEQ ID NO:66, SEQ ID NO:67 and SEQ ID NO:68, SEQ ID NO:69 and SEQ ID NO:70, SEQ ID NO:71 and SEQ ID NO:72, SEQ ID NO:73 and SEQ ID NO:74, SEQ ID NO:75 and SEQ ID NO:76, SEQ ID NO:77 and SEQ ID NO:78, SEQ ID NO:79 and SEQ ID NO:80, SEQ ID NO:81 and SEQ ID NO:82, SEQ ID NO:83 and SEQ ID NO:84, SEQ ID NO:85 and SEQ ID NO:86, SEQ ID NO:87 and SEQ ID NO:88, SEQ ID NO:89 and SEQ ID NO:90, SEQ ID NO:91 and SEQ ID NO:92, SEQ ID NO:93 and SEQ ID NO:94, SEQ ID NO:95 and SEQ ID NO:96, SEQ ID NO:97 and SEQ ID NO:98, SEQ ID NO:99 and SEQ ID NO:100, SEQ ID NO:101 and SEQ ID NO:102, SEQ ID NO:103 and SEQ ID NO:104, SEQ ID NO:105 and SEQ ID NO:106, SEQ ID NO:107 and SEQ ID NO:108, SEQ ID NO:109 and SEQ ID NO:110, SEQ ID NO:111 and SEQ ID NO:112, SEQ ID NO:113 and SEQ ID NO:114, SEQ ID NO:115 and SEQ ID NO:116, SEQ ID NO:117 and SEQ ID NO:118, SEQ ID NO:119 and SEQ ID NO:120, SEQ ID NO:121 and SEQ ID NO:122, SEQ ID NO:123 and SEQ ID NO:124, SEQ ID NO:125 and SEQ ID NO:126, SEQ ID NO:127 and SEQ ID NO:128, SEQ ID NO:129 and SEQ ID NO:130, SEQ ID NO:131 and SEQ ID NO:132, SEQ ID NO:133 and SEQ ID NO:134, SEQ ID NO:135 and SEQ ID NO:136, SEQ ID NO:137 and SEQ ID NO:138, SEQ ID NO:139 and SEQ ID NO:140, SEQ ID NO:141 and SEQ ID NO:142, SEQ ID NO:143 and SEQ ID NO:144, SEQ ID NO:145 and SEQ ID NO:146, SEQ ID NO:147 and SEQ ID NO:148, SEQ ID NO:149 and SEQ ID NO:150, SEQ ID NO:151 and SEQ ID NO:152, SEQ ID NO:153 and SEQ ID NO:154, SEQ ID NO:155 and SEQ ID NO:156, SEQ ID NO:157 and SEQ ID NO:158, SEQ ID NO:159 and SEQ ID NO:160, SEQ ID NO:161 and SEQ ID NO:162, SEQ ID NO:163 and SEQ ID NO:164, SEQ ID NO:165 and SEQ ID NO:166, SEQ ID NO:167 and SEQ ID NO:168, SEQ ID NO:169 and SEQ ID NO:170, SEQ ID NO:171 and SEQ ID NO:172, SEQ ID NO:173 and SEQ ID NO:174, SEQ ID NO:175 and SEQ ID NO:176, SEQ ID NO:177 and SEQ ID NO:178, SEQ ID NO:179 and SEQ ID NO:180, SEQ ID NO:181 and SEQ ID NO:182, SEQ ID NO:183 and SEQ ID NO:184, SEQ ID NO:185 and SEQ ID NO:186, SEQ ID NO:187 and SEQ ID NO:188, SEQ ID NO:189 and SEQ ID NO:190, SEQ ID NO:191 and SEQ ID NO:192, SEQ ID NO:193 and SEQ ID NO:194, SEQ ID NO:195 and SEQ ID NO:196, SEQ ID NO:197 and SEQ ID NO:198, and SEQ ID NO:199 and SEQ ID NO:200, respectively.

12. The process of claim 7, wherein the loci of the Alu elements comprise multiple loci selected from the group consisting of ACE, APO, B65, COL3A1, HS2.43, HS4.32, HS4.65, HS4.75, PV92, Sb22777/Sb19.12, Sb23467/Sb19.3, TPA25, Ya5NBC102, Ya5NBC120, Ya5NBC123, Ya5NBC132, Ya5NBC135, Ya5NBC147, Ya5NBC148, Ya5NBC150, Ya5NBC157, Ya5NBC159, Ya5NBC171, Ya5NBC182, Ya5NBC208, Ya5NBC212, Ya5NBC216, Ya5NBC221, Ya5NBC237, Ya5NBC239, Ya5NBC241, Ya5NBC242, Ya5NBC27, Ya5NBC311, Ya5NBC327, Ya5NBC333, Ya5NBC335, Ya5NBC345, Ya5NBC347, Ya5NBC351, Ya5NBC354, Ya5NBC45, Ya5NBC51, Ya5NBC54, Ya5NBC61, Ya5NBC96, Yb8NBC106, Yb8NBC120, Yb8NBC125, Yb8NBC13, Yb8NBC146, Yb8NBC148, Yb8NBC157, Yb8NBC181, Yb8NBC192, Yb8NBC201, Yb8NBC207, Yb8NBC227, Yb8NBC237, Yb8NBC243, Yb8NBC405, Yb8NBC412, Yb8NBC419, Yb8NBC420, Yb8NBC435, Yb8NBC437, Yb8NBC441, Yb8NBC450, Yb8NBC461, Yb8NBC463, Yb8NBC466, Yb8NBC479, Yb8NBC480, Yb8NBC485, Yb8NBC49, Yb8NBC5, Yb8NBC505, Yb8NBC516, Yb8NBC547, Yb8NBC568, Yb8NBC576, Yb8NBC585, Yb8NBC589, Yb8NBC596, Yb8NBC597, Yb8NBC598, Yb8NBC605, Yb8NBC622, Yb8NBC636, Yb8NBC65, Yb8NBC77, Yb8NBC80, Yb8NBC93, Yb9NBC10, Yb9NBC50, Yc1NBC2, Yc1NBC35, Yc1NBC53, Yc1NBC63, and Yc1RG68.

13. The process of claim 12, wherein the loci of ACE, APO, B65, COL3A1, HS2.43, HS4.32, HS4.65, HS4.75, PV92, Sb22777/Sb19.12, Sb23467/Sb19.3, TPA25, Ya5NBC102, Ya5NBC120, Ya5NBC123, Ya5NBC132, Ya5NBC135, Ya5NBC147, Ya5NBC148, Ya5NBC150, Ya5NBC157, Ya5NBC159, Ya5NBC171, Ya5NBC182, Ya5NBC208, Ya5NBC212, Ya5NBC216, Ya5NBC221, Ya5NBC237, Ya5NBC239, Ya5NBC241, Ya5NBC242, Ya5NBC27, Ya5NBC311, Ya5NBC327, Ya5NBC333, Ya5NBC335, Ya5NBC345, Ya5NBC347, Ya5NBC351, Ya5NBC354, Ya5NBC45, Ya5NBC51, Ya5NBC54, Ya5NBC61, Ya5NBC96, Yb8NBC106, Yb8NBC120, Yb8NBC125, Yb8NBC13, Yb8NBC146, Yb8NBC148, Yb8NBC157, Yb8NBC181, Yb8NBC192, Yb8NBC201, Yb8NBC207, Yb8NBC227, Yb8NBC237, Yb8NBC243, Yb8NBC405, Yb8NBC412, Yb8NBC419, Yb8NBC420, Yb8NBC435, Yb8NBC437, Yb8NBC441, Yb8NBC450, Yb8NBC461, Yb8NBC463, Yb8NBC466, Yb8NBC479, Yb8NBC480, Yb8NBC485, Yb8NBC49, Yb8NBC5, Yb8NBC505, Yb8NBC516, Yb8NBC547, Yb8NBC568, Yb8NBC576, Yb8NBC585, Yb8NBC589, Yb8NBC596, Yb8NBC597, Yb8NBC598, Yb8NBC605, Yb8NBC622, Yb8NBC636, Yb8NBC65, Yb8NBC77, Yb8NBC80, Yb8NBC93, Yb9NBC10, Yb9NBC50, Yc1NBC2, Yc1NBC35, Yc1NBC53, Yc1NBC63, and Yc1RG68 are amplified by using oligonucleotide primer pairs of SEQ ID NO:1 and SEQ ID NO:2, SEQ ID NO:3 and SEQ ID NO:4, SEQ ID NO:5 and SEQ ID NO:6, SEQ ID NO:7 and SEQ ID NO:8, SEQ ID NO:9 and SEQ ID NO:10, SEQ ID NO:11 and SEQ ID NO:12, SEQ ID NO:13 and SEQ ID NO:14, SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:23 and SEQ ID NO:24, SEQ ID NO:25 and SEQ ID NO:26, SEQ ID NO:27 and SEQ ID NO:28, SEQ ID NO:29 and SEQ ID NO:30, SEQ ID NO:31 and SEQ ID NO:32, SEQ ID NO:33 and SEQ ID NO:34, SEQ ID NO:35 and SEQ ID NO:36, SEQ ID NO:37 and SEQ ID NO:38, SEQ ID NO:39 and SEQ ID NO:40, SEQ ID NO:41 and SEQ ID NO:42, SEQ ID NO:43 and SEQ ID NO:44, SEQ ID NO:45 and SEQ ID NO:46, SEQ ID NO:47 and SEQ ID NO:48, SEQ ID NO:49 and SEQ ID NO:50, SEQ ID NO:51 and SEQ ID NO:52, SEQ ID NO:53 and SEQ ID NO:54, SEQ ID NO:55 and SEQ ID NO:56, SEQ ID NO:57 and SEQ ID NO:58, SEQ ID NO:59 and SEQ ID NO:60, SEQ ID NO:61 and SEQ ID NO:62, SEQ ID NO:63 and SEQ ID NO:64, SEQ ID NO:65 and SEQ ID NO:66, SEQ ID NO:67 and SEQ ID NO:68, SEQ ID NO:69 and SEQ ID NO:70, SEQ ID NO:71 and SEQ ID NO:72, SEQ ID NO:73 and SEQ ID NO:74, SEQ ID NO:75 and SEQ ID NO:76, SEQ ID NO:77 and SEQ ID NO:78, SEQ ID NO:79 and SEQ ID NO:80, SEQ ID NO:81 and SEQ ID NO:82, SEQ ID NO:83 and SEQ ID NO:84, SEQ ID NO:85 and SEQ ID NO:86, SEQ ID NO:87 and SEQ ID NO:88, SEQ ID NO:89 and SEQ ID NO:90, SEQ ID NO:91 and SEQ ID NO:92, SEQ ID NO:93 and SEQ ID NO:94, SEQ ID NO:95 and SEQ ID NO:96, SEQ ID NO:97 and SEQ ID NO:98, SEQ ID NO:99 and SEQ ID NO:100, SEQ ID NO:101 and SEQ ID NO:102, SEQ ID NO:103 and SEQ ID NO:104, SEQ ID NO:105 and SEQ ID NO:106, SEQ ID NO:107 and SEQ ID NO:108, SEQ ID NO:109 and SEQ ID NO:110, SEQ ID NO:110 and SEQ ID NO:112, SEQ ID NO:113 and SEQ ID NO:114, SEQ ID NO:115 and SEQ ID NO:116, SEQ ID NO:117 and SEQ ID NO:118, SEQ ID NO:119 and SEQ ID NO:120, SEQ ID NO:121 and SEQ ID NO:122, SEQ ID NO:123 and SEQ ID NO:124, SEQ ID NO:125 and SEQ ID NO:126, SEQ ID NO:127 and SEQ ID NO:128, SEQ ID NO:129 and SEQ ID NO:130, SEQ ID NO:131 and SEQ ID NO:132, SEQ ID NO:133 and SEQ ID NO:134, SEQ ID NO:135 and SEQ ID NO:136, SEQ ID NO:137 and SEQ ID NO:138, SEQ ID NO:139 and SEQ ID NO:140, SEQ ID NO:141 and SEQ ID NO:142, SEQ ID NO:143 and SEQ ID NO:144, SEQ ID NO:145 and SEQ ID NO:146, SEQ ID NO:147 and SEQ ID NO:148, SEQ ID NO:149 and SEQ ID NO:150, SEQ ID NO:151 and SEQ ID NO:152, SEQ ID NO:153 and SEQ ID NO:154, SEQ ID NO:155 and SEQ ID NO:156, SEQ ID NO:157 and SEQ ID NO:158, SEQ ID NO:159 and SEQ ID NO:160, SEQ ID NO:161 and SEQ ID NO:162, SEQ ID NO:163 and SEQ ID NO:164, SEQ ID NO:165 and SEQ ID NO:166, SEQ ID NO:167 and SEQ ID NO:168, SEQ ID NO:169 and SEQ ID NO:170, SEQ ID NO:171 and SEQ ID NO:172, SEQ ID NO:173 and SEQ ID NO:174, SEQ ID NO:175 and SEQ ID NO:176, SEQ ID NO:177 and SEQ ID NO:178, SEQ ID NO:179 and SEQ ID NO:180, SEQ ID NO:181 and SEQ ID NO:182, SEQ ID NO:183 and SEQ ID NO:184, SEQ ID NO:185 and SEQ ID NO:186, SEQ ID NO:187 and SEQ ID NO:188, SEQ ID NO:189 and SEQ ID NO:190, SEQ ID NO:191 and SEQ ID NO:192, SEQ ID NO:193 and SEQ ID NO:194, SEQ ID NO:195 and SEQ ID NO:196, SEQ ID NO:197 and SEQ ID NO:198, and SEQ ID NO:199 and SEQ ID NO:200 respectively.

14. The process of claim 5, wherein the amplification step is performed by using whole genome amplification technologies.

15. The process of claim 5, wherein the amplification step comprises using a multiplex polymerase chain reaction system.

16. The process of claim 6, wherein the step of determining the human geographic origin further comprises the step of using a STRUCTURE program.

17. A kit for determining an ancestry of an unknown DNA sample, comprising:
oligonucleotide primers that enable detection of Alu elements; and
reagents adapted for determining human geographic origin of an unknown human DNA sample using multi-locus genotypes from Alu insertion polymorphisms.

18. The kit of claim 17, further comprising reagents for extracting and isolating DNA from the sample.

19. The kit of claim 17, wherein said oligonucleotide primers comprises oligonucleotide primer pairs selected from the group consisting of SEQ ID NO:1 and SEQ ID NO:2, SEQ ID NO:3 and SEQ ID NO:4, SEQ ID NO:5 and SEQ ID NO:6, SEQ ID NO:7 and SEQ ID NO:8, SEQ ID NO:9 and SEQ ID NO:10, SEQ ID NO:11 and SEQ ID NO:12, SEQ ID NO:13 and SEQ ID NO:14, SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:23 and SEQ ID NO:24, SEQ ID NO:25 and SEQ ID NO:26, SEQ ID NO:27 and SEQ ID NO:28, SEQ ID NO:29 and SEQ ID NO:30, SEQ ID NO:31 and SEQ ID NO:32, SEQ ID NO:33 and SEQ ID NO:34, SEQ ID NO:35 and SEQ ID NO:36, SEQ ID NO:37 and SEQ ID NO:38, SEQ ID NO:39 and SEQ ID NO:40, SEQ ID NO:41 and SEQ ID NO:42, SEQ ID NO:43 and SEQ ID NO:44, SEQ ID NO:45 and SEQ ID NO:46, SEQ ID NO:47 and SEQ ID NO:48, SEQ ID NO:49 and SEQ ID NO:50, SEQ ID NO:51 and SEQ ID NO:52, SEQ ID NO:53 and SEQ ID NO:54, SEQ ID NO:55 and SEQ ID NO:56, SEQ ID NO:57 and SEQ ID NO:58, SEQ ID NO:59 and SEQ ID NO:60, SEQ ID NO:61 and SEQ ID NO:62, SEQ ID NO:63 and SEQ ID NO:64, SEQ ID NO:65 and SEQ ID NO:66, SEQ ID NO:67 and SEQ ID NO:68, SEQ ID NO:69 and SEQ ID NO:70, SEQ ID NO:71 and SEQ ID NO:72, SEQ ID NO:73 and SEQ ID NO:74, SEQ ID NO:75 and SEQ ID NO:76, SEQ ID NO:77 and SEQ ID NO:78, SEQ ID NO:79 and SEQ ID NO:80, SEQ ID NO:81 and SEQ ID NO:82, SEQ ID NO:83 and SEQ ID NO:84, SEQ ID NO:85 and SEQ ID NO:86, SEQ ID NO:87 and SEQ ID NO:88, SEQ ID NO:89 and SEQ ID NO:90, SEQ ID NO:91 and SEQ ID NO:92, SEQ ID NO:93 and SEQ ID NO:94, SEQ ID NO:95 and SEQ ID NO:96, SEQ ID NO:97 and SEQ ID NO:98, SEQ ID NO:99 and SEQ ID NO:100, SEQ ID NO:101 and SEQ ID NO:102, SEQ ID NO:103 and SEQ ID NO:104, SEQ ID NO:105 and SEQ ID NO:106, SEQ ID NO:107 and SEQ ID NO:108, SEQ ID NO:109 and SEQ ID NO:110, SEQ ID NO:111 and SEQ ID NO:112, SEQ ID NO:113 and SEQ ID NO:114, SEQ ID NO:115 and SEQ ID NO:116, SEQ ID NO:117 and SEQ ID NO:118, SEQ ID NO:119 and SEQ ID NO:120, SEQ ID NO:121 and SEQ ID NO:122, SEQ ID NO:123 and SEQ ID NO:124, SEQ ID NO:125 and SEQ ID NO:126, SEQ ID NO:127 and SEQ ID NO:128, SEQ ID NO:129 and SEQ ID NO:130, SEQ ID NO:131 and SEQ ID NO:132, SEQ ID NO:133 and SEQ ID NO:134, SEQ ID NO:135 and SEQ ID NO:136, SEQ ID NO:137 and SEQ ID NO:138, SEQ ID NO:139 and SEQ ID NO:140, SEQ ID NO:141 and SEQ ID NO:142, SEQ ID NO:143 and SEQ ID NO:144, SEQ ID NO:145 and SEQ ID NO:146, SEQ ID NO:147 and SEQ ID NO:148, SEQ ID NO:149 and SEQ ID NO:150, SEQ ID NO:151 and SEQ ID NO:152, SEQ ID NO:153 and SEQ ID NO:154, SEQ ID NO:155 and SEQ ID NO:156, SEQ ID NO:157 and SEQ ID NO:158, SEQ ID NO:159 and SEQ ID NO:160, SEQ ID NO:161 and SEQ ID NO:162, SEQ ID NO:163 and SEQ ID NO:164, SEQ ID NO:165 and SEQ ID NO:166, SEQ ID NO:167 and SEQ ID NO:168, SEQ ID NO:169 and SEQ ID NO:170, SEQ ID NO:171 and SEQ ID NO:172, SEQ ID NO:173 and SEQ ID NO:174, SEQ ID NO:175 and SEQ ID NO:176, SEQ ID NO:177 and SEQ ID NO:178, SEQ ID NO:179 and SEQ ID NO:180, SEQ ID NO:181 and SEQ ID NO:182, SEQ ID NO:183 and SEQ ID NO:184, SEQ ID NO:185 and SEQ ID NO:186, SEQ ID NO:187 and SEQ ID NO:188, SEQ ID NO:189 and SEQ ID NO:190, SEQ ID NO:191 and SEQ ID NO:192, SEQ ID NO:193 and SEQ ID NO:194, SEQ ID NO:195 and SEQ ID NO:196, SEQ ID NO:197 and SEQ ID NO:198, and SEQ ID NO:199 and SEQ ID NO:200.

20. The kit of claim 17, wherein said oligonucleotide primers that enable detection of Alu elements are primers for multiple Alu insertion polymorphisms.
